(19) United States
(12) Patent Application Publication
(10) Pub. No.: US 2010/0233295 A1
(43) Pub. Date: Sep. 16, 2010

(54) AQUEOUS MOISTURIZERS AND LUBRICANTS AND USES THEREOF

(76) Inventors: Vibha Gupta, Pinecrest, FL (US); Vineet Gupta, Pinecrest, FL (US); Boris Nikolic, Charlestown, MA (US)

Correspondence Address: Duane Morris LLP - Boston;IP Department, SUITE 500, 470 ATLANTIC AVENUE, BOSTON, MA, 02210-2243, US

(21) Appl. No.: 12/281,295

(22) PCT Filed: Feb. 28, 2007
(86) PCT No.: PCT/US07/05407

§ 371 Date: Oct. 8, 2009

Related U.S. Application Data
(63) Continuation-in-part of application No. 11/364,200, filed on Mar. 1, 2006.
Application No. 11/364,200 is a continuation of application No. 60/657,440, filed on Mar. 2, 2005.

(51) Int. Cl. A61K 33/14; A61K 33/00; A61K 31/7088; A61K 31/715; A61K 38/00; A61K 38/14; A61P 15/00

(52) U.S. Cl. 424/678; 424/680; 424/699; 424/700; 424/718; 514/44 R; 514/54; 514/2; 514/12; 424/679; 424/681

(57) ABSTRACT

This application relates to water-based personal moisturizers and lubricants that relive vaginal dryness. These compositions are non-spermicidal, sperm- and egg-friendly, and in various embodiments may mimic biological fluids, enhance sperm survival and motility, promote binding of sperm to eggs, and/or facilitate the process of fertilization. Related articles, systems, and methods of preparation and use of the compositions are also provided.

### TECHNICAL FIELD

The present invention relates generally to compositions for promoting in vivo and/or in vitro survival of gametes, improved function of sperm, oocyte, embryo, cell and tissue, and increased fertilization potential of sperm and oocytes, and to systems, articles and methods of preparation relating to such compositions.

### BACKGROUND OF THE INVENTION

Sexual difficulties, as characterized as the total or partial inability to participate in one or more stages of the sexual act, are an impediment for couples trying to conceive (TTC). One cause of sexual difficulty is the lack of vaginal lubrication. When the naturally lubricating physiological fluid or mucus secreted in the vagina is absent during vaginal intercourse, vaginal tissue can become dry and irritated and may cause discomfort, pain and sometimes bleeding. Contributors to this condition include the nature of the relationship between partners, insufficient excitement and stimulation, hormonal changes, and side effects of certain medicaments. Irritation from contraceptive creams and foams may also cause dryness, as can fear and anxiety about sex.

A number of different lubricant compositions to address vaginal dryness are known in the art. However, most over-the-counter (OTC) lubricants that are currently available, either by design or coincidence, reduce sperm viability or motility or both (18-22), and may also prevent contact between sperm and egg. Commonly available lubricants, such as K-Y Jelly and Vaseline, are undesirable for couples wishing to conceive due to their poor water solubility, poor consistency, and above all, because they are spermicidal (18-22). Such lubricants can harm sperm and oocytes, decrease sperm motility, and prevent binding of sperm with egg, thereby hindering the process of fertilization.

One component of some available vaginal lubricants, ethylenediaminetetraacetic acid (EDTA), added to prolong the shelf life of the composition, is harmful to cell viability, chelating and sequestering ions essential for cell function. Poly- and oligosaccharides in some traditional lubricant solutions also inhibit fertilization, interfering with recognition of egg by sperm. Furthermore, available lubricant compositions typically lack agents that favor or enhance the fertilization process. In sum, many existing lubricant compositions are hostile to gametes and disfavor fertilization; accordingly there is a need for compositions suitable for couples trying to conceive.

### SUMMARY OF THE INVENTION

Compositions, methods of preparation and various applications of water-based media, solutions, moisturizers and lubricants are provided. The present invention also provides articles, systems, and methods of preparation and use of the compositions.

The water-based compositions comprise an aqueous solution with a buffered pH and osmolality that approximates biological fluids. In a preferred embodiment, such water-based compositions are useful as non-spermicidal vaginal lubricants that are sperm friendly, egg friendly, and/or facilitate fertilization. The water-based solutions may further comprise sperm and cell viability-maintaining agents, energy-boosting agents for sperm and cells, antioxidants and oxygen scavengers, fertilization facilitation agents, and/or embryo attachment facilitating agents.

In yet another aspect, methods and compositions are provided for improving human and other animal reproduction using natural or artificial means. The compositions can also be used in combination with articles and systems to facilitate fertilization.

The present invention further provides compositions, articles, systems and methods of preparation and use of compositions that do not harm cells, tissues or organs, and/or improve their viability and/or function during culture, storage, transportation, and/or in vivo and/or in vitro use.

### BRIEF DESCRIPTION OF THE FIGURES

The following figures depict certain illustrative embodiments of the invention in which like reference numerals refer to like elements. These depicted embodiments are to be understood as illustrative of the invention and not as limiting in any way.

FIG. 1. is a depiction of a lubricated sheath designed to be placed over the penis for use by couples trying to conceive.

FIG. 2. is a depiction of a lubricated sheath designed to be placed within the vagina for use by couples trying to conceive.

### DETAILED DESCRIPTION OF THE INVENTION

For couples trying to conceive, the success of fertilization can be hindered by non-natural sexual aids (spermicidal lubricants) as well as inherent biological and/or physiological conditions in the sexual organs. In semen, spermatozoa encounter a basic pH, sometimes as basic as pH 7.8-8.2, whereas the pH in the vagina is typically more acidic, e.g., around 4.5, a pH which favors normal vaginal flora but is harmful to sperm. Prior to entering the female tract, the sperm are stored within the cauda epididymis in a functionally inactive state, immotile and incapable of interacting with eggs (1-16). In the cauda epididymis, stored sperm are kept viable by the presence of ions, energy substrates, and nutrients in a pH- and tonically balanced solution. This environment contains a high concentration of potassium ions, low sodium ions and very low bicarbonate ions (24).

Inactive sperm are transformed into free-swimming and functional cells that are capable of fertilizing eggs in a series of biochemical steps, aided by changes in the biological milieu. Sperm capacitation involves, cholesterol efflux from sperm membranes, cAMP-dependent signaling (which requires extracellular bicarbonate ions), and the elevation of intracellular pH and bicarbonate levels with the associated stimulation of cAMP production (which is linked to the control of flagellar motility). Additionally, progesterone may also regulate some aspects of capacitation and lower than physiological levels of potassium ions also favor capacitation (23). The composition of capacitation-inducing oviductal fluid and media comprises a high concentration of various ions (such as sodium, calcium and bicarbonate) and low concentrations of others (such as potassium (17)). In certain embodiments of the invention, compositions include a balanced salt solution to improve viability, motility and/or mucus penetration of sperm after transfer to a female to improve the chances of fertilization.

Capacitated sperm penetrate the cumulus oophorus (assisted by a cell surface hyaluronidase), contact the zona pellucida, and undergo an acrosome reaction (which is calcium-dependent). Sperm adhesion to the zona pellucida is based on binding between cell surface receptors and ligands on the cells (such as integrins) and on protein-carbohydrate recognition processes. After completion of the acrosome reaction, sperm penetrate the zona pellucida, subsequently contacting and fusing with the plasma membrane of the egg, leading to fertilization. Furthermore, motion of the sperm during this process is energy intensive, and is aided by energy substrates, including pyruvate, lactate and glucose. In certain embodiments of the invention, compositions include sperm energy-boosting agents, to improve viability, motility and/or mucus penetration of sperm.

Initial sperm-zona pellucida binding is mediated by ZP3, a constituent glycoprotein of the zona pellucida, and involves protein-carbohydrate recognition and interaction. For example, the sperm surface receptors associate with O-linked oligosaccharides from protein ZP3 on the zona pellucida. Thus, presence of foreign oligo- and poly-saccharides can interfere with this recognition, thereby negatively impacting the process of fertilization. In certain embodiments of the invention, the composition is free or substantially free of poly- and oligo-saccharides, e.g., that interfere with the protein-carbohydrate egg-sperm recognition process.

Furthermore, one of the last steps prior to a successful fertilization is the binding and fusing of sperm that have penetrated the zona pellucida with the cell membrane of the egg. Certain preservatives such as EDTA can interfere with successful fertilization processes by sequestering beneficial ions. In certain embodiments of the invention, compositions comprise agents that enhance the binding of sperm cells with oocytes and are free or substantially free of certain detrimental preservatives, such as EDTA.

In addition, certain cell surface proteins that are important for binding between sperm and egg, such as integrins, are naturally present in an inactive conformation on the cells and therefore do not bind their ligands until activated by signals from the environment. In certain embodiments of the invention, compositions comprise agents that enhance the binding of sperm cells with oocytes by activating integrin receptors. Inclusion of such agents in lubricant compositions may prime or activate cell surface receptors on sperm and egg for binding to each other, thereby enhancing their fertilization potential.

For a number of years now, the use of artificial insemination and of assisted reproduction techniques (ART) has allowed physicians to treat fertility issues in individuals. These techniques have also benefited from methods for storing cells, sperm, oocytes or embryos for use at different times and/or locations. The steps and methods involved, some of which are optional, include: collection of cells, sperm, egg or embryo from humans and/or other animals; washing the collected samples (for example, washing semen to isolate the sperm-rich fraction, or washing eggs); subsequent processing to obtain viable and functional cells; culturing of cells; in vitro fertilization to develop embryos; storage of cells or embryos (in extended culture, refrigerated or cryogenic state) for later use; reprocessing prior to transfer to female; and transfer to female in order to establish pregnancy. Improved compositions for semen and sperm collection that contain viability-maintaining agents or fertilization-enhancing agents, for example, would improve the chances of sperm-egg binding, thereby improving the fertilization potential. Similarly, use of compositions containing viability-maintaining agents or fertilization and/or other function-enhancing agents in sperm wash and sperm isolation media would also improve the chances of sperm-egg binding, thereby improving the fertilization potential. Similarly, use of compositions containing viability-maintaining agents or fertilization and other function enhancing agents in in vitro fertilization media would also improve the chances of sperm-egg binding, thereby improving the fertilization potential. The present invention further provides compositions that do not harm cells, sperm, oocytes, embryos, tissues or organs and furthermore may improve their viability and function during culture, during storage, transportation and during in vivo and in vitro use. Additionally, compositions are provided for use in various artificial insemination and assisted reproduction techniques in humans and animals.

Additionally, certain medical and/or physical conditions make it advisable or even necessary to use vaginal and/or surgical lubricants. For example, compositions of the invention could be used as part of an injection for relieving osteoarthritis pain. Some such treatments are administered as a course of injections into the knee joint and are believed to supplement the viscosity of the joint fluid thereby lubricating the joint, cushioning the joint and producing an analgesic effect and potentially improving the viability of cartilage cells. Alternately compositions of the invention could be tailored for use during eye surgery (e.g. corneal transplantation, cataract surgery, glaucoma surgery and surgery to repair retinal detachment), offering lubricating and/or cell viability enhancing effects. Alternately, compositions of the invention could be incorporated in biomaterial scaffolds to engineer tissue growth aided by the cell viability-maintaining agents of the current invention. In another embodiment of the invention, lubricant compositions with cell viability-maintaining agents are provided for a variety of medical uses.

One of the qualities of a lubricant that facilitates the process of fertilization is that it not reduce the viability of the gametes. A second desirable characteristic is that the composition not negatively impact the motility, penetration and/or fertilization potential of sperm or the fertilization potential of oocyte. In addition, the composition should also not harm the oocyte or the fertilized embryo. Such compositions are preferably also non-toxic to the oocyte and the fertilized embryo. The compositions of the invention are particularly useful as lubricants for use prior to or during coitus.

The present invention provides a variety of compositions that are compatible with sperm, oocytes, embryos, cells and tissues and are non-toxic. Additionally, such compositions may even improve sperm and oocyte function and survival as well as the chance of successful fertilization. Thus, these described compositions are useful for couples trying to conceive (whether naturally or using any of a variety of assisted reproduction techniques). The invention provides compositions, articles, systems, methods of preparation and use for various applications of water-based media, solutions, moisturizers, and lubricants.

As used herein, the term “viability-maintaining agents”, unless otherwise specified, refers to agents that are non-toxic to cell, sperm, oocyte, embryo, or tissue and maintain or increase their viability. Examples of viability-maintaining agents include, but are not limited to, naturally occurring or synthetic ions and salts, such as calcium, sodium, potassium or magnesium ions and salts. Viability-maintaining agent or agents also include other ions, salts, lipids, small molecules, carbon monoxide, carbon dioxide, nitric oxide, nucleosides, nucleotides, sugars, peptides, proteins, and chemical, functional and/or physiological equivalents thereof.

As used herein, the term “aqueous lubricant base”, unless otherwise specified, refers to water-based compositions containing a lubricious agent in an aqueous balanced salt solution. Examples of lubricious agents include, but are not limited to, glycerol, HISPAGEL (glyceryl polyacrylates), arabinogalactan, PCAGH (polysaccharides containing arabinose, galactose, and/or hexuronic acid), dextran, polyacrylic acid, carbomer (homopolymers of acrylic acid cross-linked with an allyl ether pentaerythritol, allyl ether of sucrose, or allyl ether of propylene), polyethylene oxide, Pluronic (copolymers of ethylene oxide and propylene oxide, e.g., Pluronic-127), methylcellulose, hydroxymethyl cellulose, hydroxyethyl cellulose, hydroxypropyl methylcellulose, polyethylene glycol, propylene glycol, hydroxypropyl guar (2-hydroxypropyl ether), plant oils, methylparaben, proteins, nucleic acids, petroleum jelly, combinations thereof or other agents or combinations that are chemically, functionally, or physiologically equivalent or similar.

As used herein, the term “balanced salt solutions”, unless otherwise specified, refers to aqueous solutions that have biologically suitable pH and osmolality. Examples of pH buffering agents include, but are not limited to, salts of phosphates, borates, citrates, ascorbates, carbonates, bicarbonates, TRIS (Trihydroxymethylaminoethane), HEPES (4-(2-hydroxyethyl)-1-piperazineethanesulfonic acid), or a mixture thereof. Examples of osmotically active agents useful for balancing osmolality of a composition include, but are not limited to, sodium ions, potassium ions, chloride ions, bicarbonate ions, glucose, sucrose, peptides, proteins, a combination thereof or other agents that are chemically, functionally, or physiologically equivalent or similar.

As used herein, the term “enhanced physiological function”, unless otherwise specified, refers to an improvement in the potential of cell, sperm, oocyte, embryo or tissue to perform their natural function. Examples of enhanced physiological function include, but are not limited to, increase in the potential of sperm to fertilize, increase in the potential of oocyte to be fertilized, increase in the potential of embryo to develop, and increase in the potential of a fertilized embryo to attach to the uterine wall.

As used herein, the term “sperm activation”, unless otherwise specified, refers to the process of converting non-motile, functionally inactive sperm to a state that is capable of fertilization. Examples of sperm activation processes include, but are not limited to, decreasing sperm immotility and inducing or improving sperm capacitation. Examples of sperm activation agents include, but are not limited to, ions (such as bicarbonate, sodium, calcium, magnesium and manganese), salts, hyaluronidase (such as PH-20), albumin, high-density lipoprotein, progesterone, peptides, nucleosides, nucleotides, cyclic AMP, small molecules, proteins, antibodies, chemokine, cytokines, prostaglandins, caffeine, aspirin, carbon monoxide, carbon dioxide, nitric oxide, a combination thereof or other agents or combinations that are chemically, functionally, or physiologically equivalent or similar.

As used herein, the term “energy-boosting agent”, unless otherwise specified, refers to natural or synthetic substances that provide energy substrates to cell, sperm, oocyte, embryo or tissue. Examples of energy-boosting agents include, but are not limited to, adenosine triphosphate (ATP), pyruvate, glucose, lactose, other sugars, lactate, glycerolphosphorylcholine, other lipids, carnitine, amino acids, peptides, proteins, a combination thereof or other agents or combinations that are chemically, functionally, or physiologically equivalent or similar.

As used herein, the term “scavenger”, unless otherwise specified, refers to natural or synthetic substances that react with free radicals and/or prevent free radicals from causing damage to deoxyribonucleic acid (DNA), ribonucleic acid (RNA), peptides, proteins, or the membrane, organelles, structure and/or function of cells, sperm, oocytes, embryos, and/or tissue. Examples of scavengers include, but are not limited to, vitamin E, Vitamin C, niacin, riboflavin, niacinamide, glutathione, NADH, other anti-oxidants, a combination thereof or other agents or combinations that are chemically, functionally, or physiologically equivalent or similar.

As used herein, the term “fertilization facilitator”, unless otherwise specified, refers to natural or synthetic substances that facilitate the process of fertilization or remove one or more agents that may hinder the process of fertilization. As an example, the fertilization facilitators may function by increasing the potential of sperm and egg surface receptors and ligands to interact. Examples of fertilization facilitators include, but are not limited to, ions (such as magnesium, manganese, bicarbonate and zinc), hyaluronidase (such as PH-20), albumin, high-density lipoprotein, progesterone, panthenol, caffeine, L-carnitine, cyclic-AMP, aspirin, activators of CD9 protein, activators of surface receptors (such as activators integrins), a combination thereof or other agents or combinations that are chemically, functionally, or physiologically equivalent or similar.

The term “reproductive tissue”, unless otherwise specified, refers to human or other animal tissues involved in the process of reproduction. Examples of reproductive tissue include, without limit, mucous, vaginal, uterine, urethral, and penile tissue, and skin surrounding vaginal and penile tissue among others.

The term “organ”, unless otherwise specified, refers to any type of human or other animal organ. Examples of organs include, without limit, reproductive organs (such as vagina, penis, uterus, ovary), kidney, heart, skin, lung, and liver, among others.

The term “cells”, unless otherwise specified, refers to any type of human or other animal cells. Examples of cells include, without limit, sperm, oocytes, and embryonic cells, among others.

The compositions and articles may be prepared and/or produced by any method, including combining the active ingredients in the appropriate amounts and concentrations. Other suitable active or inactive agents can optionally be added. The absolute weight of a given agent included in a composition can vary widely. The compositions are preferably sterile and a preferred method of sterilization is passing through a 0.2 micron filter.

### I. Lubricant and Moisturizer Compositions and Applications

The invention is partly a result of our unexpected finding that use of viability-maintaining agents and/or fertilization- and other physiological function-enhancing agents in lubricants and moisturizers improves the viability of biological cells and tissues as well as improves their function. For example, when spermatozoa are incubated in various buffers for 30 minutes and then analyzed for viability using a FACS assay (as described in Example 3), we find (Table I) that there is almost no loss in viability of spermatozoa incubated with lubricants that include divalent ions as viability-maintaining agents (Composition 1, Composition 2, and Composition 3) as compared to lubricants that don't (such as K-Y jelly, a lubricant known to be spermicidal (18-22)). An even more surprising result was our finding that even compared with a commercially available non-spermicidal lubricant (such as Pre-Seed), the addition of divalent ions as viability-maintaining agents in lubricant compositions increases sperm viability. Lubricants based on any of several other lubricious agents, including glycerol, propylene glycol, and polyethylene glycol, in combination with viability-maintaining agents gave similar results.

TABLE I

| | Relative percentage of |
|---|---|
| Lubricant | dead spermatozoa |
| K-Y Jelly | 100% |
| Pre-Seed | 44% |
| Composition 1 | 1% |
| Composition 2 | 1% |
| Composition 3 | 1% |

In certain embodiments, the compositions of the invention are free or substantially free of certain preservatives and buffers that affect viability or function of cell, sperm, oocyte, embryo or tissues, are non-spermicidal and comprise one or more of the following: viability-maintaining agents, an aqueous lubricant base, sperm activation agents, energy-boosting agents, scavengers, fertilization facilitators, preservatives that do not affect viability or function of cell, protective agents that reduce the loss of viability of stored cells, tissues or organs, other pharmaceutically useful agents and embryo implantation-enhancing agents. A composition that is substantially free of a substance has less than an amount of that substance that causes a measurable deleterious effect. Typically, that amount is less that 0.1%, less than 0.01%, or even less than 0.001% of the substance.

In certain embodiments, water-based compositions are provided, comprising one or more viability-maintaining agents in an aqueous lubricant base. The composition preferably has a pH, osmolality and/or viscosity in a suitable range for maintaining or enhancing sperm viability and/or motility. In certain embodiments, the compositions of the invention further comprise one or more agents that enhance physiological function, e.g., sperm activation agents, energy-boosting agents, scavengers, fertilization facilitators, embryo implantation facilitators, or any combination thereof.

In another aspect, the invention provides a water-based composition comprising one or more agents that enhance sperm physiological function in an aqueous lubricant base. The compositions preferably have a pH, osmolality and/or viscosity in a suitable range for maintaining or enhancing sperm viability and/or motility. In certain such embodiments, the agents that enhance physiological function include one or more of: sperm activation agents, energy-boosting agents, scavengers, fertilization facilitators, or a combination thereof. In certain embodiments, such compositions further comprise one or more viability-maintaining agents.

In certain embodiments, compositions are provided that are free or substantially free of certain preservatives and/or buffers that reduce the viability and/or function of cells, sperm, oocytes, embryos, or tissues. Examples of such preservatives include, without limit, EDTA, cyclic-RGD peptide and certain other proteins, glycoproteins, sugars or oligo- and poly-saccharides that inhibit cell-cell and cell-matrix interaction, such as sperm-egg recognition and penetration. Thus, in certain embodiments, the composition is free or substantially free of EDTA. In certain embodiments, the composition is free or substantially free of glycoproteins, sugars or oligo- and/or poly-saccharides that inhibit cell-cell and cell-matrix interaction, such as sperm-egg recognition and penetration. The composition of the invention preferably has a pH, osmolality and/or viscosity in a suitable range for maintaining or enhancing sperm viability and/or motility. In certain embodiments, the compositions further comprise one or more viability-maintaining agents and/or agents that enhance physiological function (e.g., sperm activation agents, energy-boosting agents, scavengers, fertilization facilitators, or a mixture thereof).

In certain embodiments, the aqueous lubricant base may be as described above, e.g., an aqueous salt solution with balanced pH and osmolality, and preferably comprises one or more of the following components: glycerol, HISPAGEL, dextran, polyacrylic acid, carbomer, polyethylene oxide, methylcellulose, hydroxyethyl cellulose, hydroxypropyl methylcellulose, polyethylene glycol, propylene glycol, PLURONIC-127, proteins, nucleic acids or petroleum jelly, or any combination thereof. In certain embodiments, the aqueous lubricant base comprises hydroxypropyl methylcellulose. In certain other embodiments, aqueous lubricant base comprises propylene glycol, glycerol, or a mixture thereof. The composition comprises between 1% and 99.999% water, preferably between 75% and 99.99% water, and most preferably between 95% and 99.9% water. In certain embodiments, the pH of the composition is in the range of 5.0 and 9.0, preferably between 7.0 and 8.5, more preferably between 7.2 and 8.0, or between 7.8 and 8.2; for example, about 7.35; the osmolality is between 200 and 700 mOsm/kg, preferably between 250 and 500 mOsm/kg, more preferably between 290 and 360 or between 300 and 400 mOsm/kg and most preferably about 320 mOsm/kg; and the viscosity, expressed as ratio with the viscosity of a balanced salt solution such as phosphate buffered saline (PBS), is between 1.0 and 5.0, preferably between 1.0 and 3.5, and most preferably between 1.0 and 2.5. Suitable pH buffering agents include phosphate salts, borate salts, citrate salts, ascorbate salts, carbonate salts, bicarbonate salts, or a combination thereof. Other buffering agents, such as TRIS, PIPES (piperazine-1,4-bis(2-ethanesulfonic acid)), HEPES and the like may be added to these solutions. In certain embodiments, sodium hydroxide is added to adjust the pH. In certain embodiments, osmolytes comprise sodium ions, potassium ions, inositol, betaine, sorbitol, peptides or glutamine. In certain embodiments, the concentration of potassium ions is low, e.g., between 0.001 micromolar (μM) and 12.5 millimolar (mM), preferably between 0.1 μM and 10 mM, and most preferably between 10 μM and 5 mM.

In certain embodiments, the viability-maintaining agent is ionic, such as calcium or magnesium ions. In other embodiments, the preferred viability-maintaining agent is selected from carbon monoxide, carbon dioxide, nitric oxide or a mixture thereof. In certain embodiments, the composition comprises one or more viability-maintaining agents at a concentration between 0.001 micromolar (μM) and 1 molar (M), preferably between 0.01 millimolar (mM) and 10 mM, and most preferably between 0.1 mM and 5 mM.

In certain embodiments, the sperm activation agent is ionic, such as calcium, magnesium, manganese, or bicarbonate ions or a combination thereof. In another aspect, the composition comprises one or more sperm activation agents, e.g., provided at a concentration between 0.001 micromolar (μM) and 1 molar (M), preferably between 0.01 μM and 50 mM, and most preferably between 1 μM and 30 mM. In certain embodiments, the sperm activation agent is ionic calcium, e.g., provided at a concentration between 10 μM and 10 mM, and preferably between 500 μM and 5 mM. In certain other embodiments, the sperm activation agent is ionic magnesium, e.g., provided at a concentration between 10 μM and 10 mM, and preferably between 500 μM and 5 mM. In certain other embodiments, the sperm activation agent is bicarbonate ion, e.g., provided at a concentration between 100 μM and 50 mM, and preferably between 10 mM and 30 mM. In certain other embodiments, the sperm activation agent is cyclic AMP, caffeine, acetylsalicylic acid (aspirin), carbon monoxide or a mixture thereof. In certain such embodiments, the sperm activation agent is caffeine. In yet other embodiments, the sperm activation agent is selected from hyaluronidase (such as PH-20), albumin, high-density lipoprotein, progesterone or a mixture thereof.

In certain embodiments, the energy-boosting agent is pyruvate, lactate or a mixture thereof, e.g., provided at a concentration between 0.0001 μM and 100 mM, preferably between 0.01 μM and 10 mM, and most preferably between 1 μM and 1 mM.

In certain embodiments, the scavenger is vitamin E, Vitamin C, niacin, riboflavin, niacinamide or a mixture thereof; the provided concentration between 0.0001 μM and 100 mM, preferably between 0.01 μM and 10 mM, and most preferably between 1 μM and 1 mM.

In certain embodiments, the preferred fertilization facilitator is chosen from magnesium ions, manganese ions, bicarbonate ions, hyaluronidase, progesterone, panthenol, caffeine, L-carnitine, cyclic-AMP or a mixture thereof. In a related embodiment, the concentration of fertilization facilitator is between 0.0001 μM and 100 mM, preferably between 0.01 μM and 10 mM, and most preferably between 1 μM and 2 mM.

In certain embodiments, the compositions of the invention are free or substantially free of preservatives that reduce viability or function of any one of: cells, sperm, oocytes, embryos or tissues. Examples of such agents include, without limit, EDTA, cyclic-RGD peptide and certain glycoproteins, sugars or oligo-saccharides that inhibit cell-cell and cell-matrix interaction. In certain embodiments, the composition is free or substantially free of EDTA. In certain embodiments, the non-desired preservatives are certain glycoproteins, sugars or oligo-saccharides that inhibit cell-cell and cell-matrix interaction.

In certain embodiments, the compositions of the invention contain preservatives that do not reduce viability or function of any one of cells, sperm, oocytes, embryos or tissues. Examples of preservatives that do not reduce viability or function of cell, sperm, oocyte, embryo or tissues include, without limit, boric acid, ascorbic acid, sodium borate, methyl paraben or a combination thereof; the preservative is provided at a concentration between 0.00001% and 10%, preferably between 0.0001% and 5%, and more preferably between 0.001% and 1%.

In certain embodiments, the compositions of the invention contain one or more other pharmaceutically useful agents including, without limit, anti-itch agents, anesthetic agents, estrogenic agents, antibiotic agents, antiviral agents, anti-fungal agents, steroids, therapeutic drugs, drug delivery vehicles and others, and including combinations thereof. Penicillin, streptomycin, gentamycin, or mixtures thereof are preferred antibiotics.

In certain embodiments, the compositions of the invention contain one or more other embryo implantation potential-enhancing agents including, without limit, hyaluronan.

In certain embodiments, compositions of the invention are non-staining, non-irritating, clear, odorless, without undesired preservatives and/or non-spermicidal.

In various embodiments, compositions of the invention are in the form of a solution, gel, foam, cream, jelly, suppository, douche, film, dissolvable film or the like. Additionally, the compositions may be packaged in a container, e.g., a sealed and/or container, such as pre-filled single-use applicators, tubes, ampoules, packages, vials, bottles (e.g., pump bottles, squeeze bottles, etc.), jars, tubs, pouches, or bags. In certain embodiments, an applicator is designed to allow minimal contact with skin, thus limiting contamination with harmful microorganisms such as yeast, bacteria and viruses. In certain embodiments, the composition may be packaged in a kit containing a tube of the lubricant composition and a device to facilitate application to the vagina. In another aspect, the kit may contain one or more tubes of the lubricant composition and other items, including without limit, instruction sheets, vitamin pills, fertility monitors, nutraceuticals and the like.

In certain embodiments, the subject compositions are used as tissue moisturizers and lubricants in humans or other animals. In certain embodiments, the compositions are useful as vaginal lubricants and moisturizers. In certain embodiments, the lubricants provided are non-spermicidal, sperm-friendly, oocyte-friendly and embryo-friendly. In certain embodiments, the lubricant compositions provided increase the fertilization-potential of sperm and of oocyte. In certain embodiments, the lubricant compositions provided increase implantation potential of fertilized embryo.

In certain embodiments, the lubricants and moisturizers are used during coitus, in various assistive reproduction techniques, such as ART, and/or in various other medical and diagnostic procedures in human and other animals.

In certain embodiments, compositions of the invention may be administered or placed in a vagina prior to or during coitus. The composition may also be administered or placed in a vagina prior to or during artificial insemination, or prior to or during an in vitro fertilization procedure. In certain embodiments, the composition may be applied or administered to a penis prior to or during coitus, or prior to or during an in vitro fertilization or artificial insemination procedure. Examples include, without limit, application or administration of the composition during semen or sperm collection to a penis prior to ejaculation of semen or sperm into a collection vessel, or collection of semen or sperm into a collection vessel containing the composition. In certain embodiments, the composition may be added to the semen or sperm collection vessel prior to, during or subsequent to sperm collection.

In certain embodiments, the compositions are used to coat, lubricate and moisturize tissues. In other embodiments, the compositions are used to coat, lubricate and moisturize, surfaces and/or articles, such as lubricated sheaths for the penis, wherein the composition is used to lubricate the inside, outside or both surfaces of a sheath. Similarly, the composition may be used as a lubricant for a medical device or hand prior to or during medical or reproductive procedures.

In certain embodiments, the methods and compositions of the invention are used in preparing dermatological creams, gels, moisturizers and lubricants to relieve dryness and irritation.

In certain embodiments, compositions of the invention are provided for use as a lubricant for delivery of a child at birth.

In certain embodiments, the compositions of the invention are used for collection, transport or storage of various biological samples, such as, without limit, cerebrospinal fluid, biopsy cells, biopsy tissue, cysts, tumors, saliva, stool, buccal swab, tissue, cells, blood, fluid or a mixture thereof. In certain such embodiments, the compositions of the invention preserve viability of the collected biological material. Similarly, the compositions of the invention may be used for culturing or extending media immediately after collection of biological samples. Similarly, the compositions of the invention may be useful as culturing or extending media for biological samples immediately following their storage in a controlled temperature, e.g., heated, refrigerated, frozen or vitrified state.

In certain embodiments, compositions of the invention are used for collection, transport or storage of microbiological flora (such as, without limit, bacteria, fungi, virus etc) from biological samples, such as, without limit, cerebrospinal fluid, biopsy cells, biopsy tissue, cysts, tumors, saliva, stool, buccal swab, cells, tissue, fluid, blood or a mixture thereof. For example, the compositions of the invention may preserve viability of the collected microflora. Similarly, the compositions of the invention are useful as culturing or extending media of the microflora immediately after collection of biological samples. Furthermore, the compositions of the invention are useful as culturing or extending media of the microflora for biological samples immediately following their storage in a refrigerated, frozen or vitrified state. For example, the microflora collected using the compositions are used in, without limit, development of assays, small molecules, therapeutics, high-throughput screenings etc.

In certain embodiments, compositions of the invention are used for collection, transport or storage of various biological organs, such as, without limit, vagina, penis, kidney, lung, heart, liver, bone, skin and the like. In certain such embodiments, the compositions of the invention preserve viability of the collected organs. In certain such embodiments, the compositions of the invention preserve physiological function of the collected organs. For example, the compositions of the invention may enhance the function of an organ post-transplantation, by reducing the rejection rate. In certain such embodiments, the compositions of the invention are useful as culturing or extending media immediately after collection of organs. In certain embodiments, the compositions of the invention are useful as culturing or extending media for organs immediately following their storage in a controlled temperature, room temperature, heated, body temperature, refrigerated, frozen or vitrified state. In certain embodiments, the compositions of the invention are useful as transportation solutions for organs. For example, the compositions of the invention may improve the viability and/or physiological function of organs during culture, during storage, transportation and during in vivo and in vitro use.

In certain embodiments, the compositions of the invention are used during medical treatments for wounds, rashes, burns, bruises, transplants, or other suitable conditions.

The compositions and articles of the invention may be prepared and/or produced by any suitable method, including combining the active ingredients in the appropriate amounts and concentrations in a container and mixing. If need be, the mixtures are heated or cooled to aid in solvation of the ingredients. The compositions are preferably sterile and the most preferred method of sterilization is passing through a 0.2 micron filter. In another aspect, the compositions of the invention also have a high degree of clarity, preferably a turbidity of less than about 2, as measured with standard turbidimetric procedures known in the art.

### II. Media Compositions and Application

The invention further provides compositions for use as media as well as related methods of use.

In certain embodiments, the media compositions are used as culture and/or extension media for sperm, oocyte, embryo, cells or tissues during their in vitro culture or extension, comprising viability-maintaining agent or agents in a balanced salt solution base. The composition of the invention preferably has a pH, osmolality and/or viscosity compatible with cell culture. In certain embodiments, the compositions of the invention further comprise one or more agents that enhance physiological function, e.g., sperm activation agents, energy-boosting agents, scavengers, fertilization facilitators or a mixture thereof. In certain embodiments, compositions of the invention are free or substantially free of certain preservatives and buffers that reduce viability or function of cell, sperm, oocyte, embryo or tissues. Examples of such preservatives include, without limit, EDTA, cyclic-RGD peptide and certain glycoproteins, sugars or oligo-saccharides that inhibit cell-cell and cell-matrix interaction.

In certain embodiments, the media compositions of the invention are used as culture and extension media for sperm, oocytes, embryos, cells or tissues during their in vitro culture or extension, and comprise one or more agents that enhance physiological function in a balanced salt solution. The media compositions of the invention preferably have a pH, osmolality and viscosity suitable for the maintenance and growth of cells and/or tissues. In certain embodiments, agents that enhance physiological function may be sperm activation agents, energy-boosting agents, scavengers, fertilization facilitators, or a combination thereof. In certain embodiments, the media compositions of the invention further comprise one or more viability-maintaining agents. In certain embodiments, compositions of the invention are free or substantially free of certain preservatives and buffers that reduce viability or function of cells, sperm, oocytes, embryos or tissues. Examples of such preservatives include, without limit, EDTA, cyclic-RGD peptide and certain glycoproteins, sugars or oligo-saccharides that inhibit cell-cell and cell-matrix interaction.

In certain embodiments, balanced salt solution base of a media composition may include, without limit, Iscove's Modified Dulbecco's Medium (IMDM), Dulbecco's Medium Eagle's Medium (DMEM), Roswell Park Memorial Institute (RPMI) media (such as RPMI 1640), Tyrode's buffered salts, Oocyte collection media, TALP, HTF, CZB, T6, Ham's F12, Earle's buffered salts, BWW, Earle's MTF, KSOM, SOF, PBS and the like. These and other balanced salt solutions are well known in the art and many are commercially available (for example, from ATCC, Fisher Scientific, Invitrogen, VitroLife etc.). Furthermore, other buffering agents, such as TRIS, PIPES, and HEPES may be added to these solutions. Additionally, certain other agents, such as serum, albumin, gelatin, vitamins, minerals, amino acids, nucleotides, sucrose, trehalose, ethanol, DMSO, hydroxypropyl methylcellulose, may also be added to the buffered salt solutions. The composition comprises between 50% and 99.999% balanced salt solution, preferably between 75% and 99.99% balanced salt solution, and most preferably between 95% and 99.9% balanced salt solution. In certain embodiments, the pH of the composition is in the range of 5.0 and 9.0, preferably between 7.0 and 8.5, preferably between 7.8 and 8.2 and preferably about 7.35; the osmolality is between 200 and 700 mOsm/kg, preferably between 250 and 500 mOsm/kg, more preferably between 300 and 350 mOsm/kg and most preferably about 320 mOsm/kg; and the viscosity, expressed as ratio with the viscosity of a balanced salt solution such as phosphate buffered saline (PBS), is between 1.0 and 5.0, preferably between 1.0 and 2.5, and most preferably between 1.0 and 1.2. In certain embodiments, suitable pH buffering agents include phosphate salts, borate salts, citrate salts, ascorbate salts, carbonate salts, bicarbonate salts, or a mixture thereof. Sodium hydroxide may be added to adjust the pH. In certain embodiments, osmolytes comprise sodium ions, potassium ions, inositol, betaine, sorbitol, peptides or glutamine. In certain embodiments, the concentration of the osmolyte potassium ions is low, between 0.001 micromolar (μM) and 12.5 millimolar (mM), preferably between 0.1 μM and 10 mM, and most preferably between 10 μM and 5 mM.

In certain embodiments, the viability-maintaining agent is ionic, e.g., calcium or magnesium ions. In certain embodiments, the media composition comprises one or more viability-maintaining agents. In certain embodiments, the viability-maintaining agent is provided at a concentration between 0.001 micromolar (μM) and 1 molar (M), preferably between 0.01 millimolar (mM) and 10 mM, and most preferably between 0.1 mM and 5 mM. In other embodiments, the preferred viability-maintaining agent is selected from carbon monoxide, carbon dioxide, nitric oxide or a mixture thereof.

In certain embodiments, the preferred sperm activation agent is ionic, and the preferred ions are calcium, magnesium, manganese, or bicarbonate. In a related aspect, the media composition comprises one or more sperm activation agents. In another aspect, the provided concentration of sperm activation agent is between 0.001 micromolar (μM) and 1 molar (M), preferably between 0.01 μM and 50 mM, and most preferably between 1 μM and 30 mM. In another aspect, the preferred sperm activation agent is ionic calcium and the provided concentration of calcium is between 10 μM and 10 mM, and preferably between 500 μM and 5 mM. In another aspect, the preferred sperm activation agent is ionic magnesium and the provided concentration of magnesium is between 10 μM and 10 mM, and preferably between 500 μM and 5 mM. In another aspect, the most preferred sperm activation agent is bicarbonate ion and the provided concentration of bicarbonate ion is between 100 μM and 50 mM, and preferably between 10 mM and 30 mM. In other embodiments, the sperm activation agent is cyclic AMP, caffeine, aspirin, carbon monoxide or a mixture thereof. In related embodiments, the preferred sperm activation agent is caffeine. In another aspect, the preferred sperm activation agent is selected from hyaluronidase (such as PH-20), albumin, high-density lipoprotein, progesterone or a mixture thereof.

In certain embodiments, the preferred energy-boosting agent is ATP, fructose, glucose, pyruvate, lactose, lactate or a mixture thereof. In related embodiments, the concentration of energy-boosting agent is between 0.0001 μM and 1 M, preferably between 0.01 μM and 100 mM, and most preferably between 1 μM and 25 mM.

In certain embodiments, the preferred scavenger is vitamin E, Vitamin C, niacin, riboflavin, niacinamide or a mixture thereof. In related embodiments, the concentration of scavenger is between 0.0001 μM and 100 mM, preferably between 0.01 μM and 10 mM, and most preferably between 1 μM and 1 mM.

In certain embodiments, the fertilization facilitator is selected from magnesium ions, manganese ions, bicarbonate ions, hyaluronidase, progesterone, panthenol, caffeine, L-carnitine, cyclic-AMP or a mixture thereof. In related embodiments, the concentration of fertilization facilitator is between 0.0001 μM and 100 mM, preferably between 0.01 μM and 10 mM, and most preferably between 1 μM and 2 mM.

In another aspect, the media composition of the invention is free or substantially free of preservatives that affect viability or function of cell, sperm, oocyte, embryo or tissues. Examples of the non-desired preservatives include, without limit, EDTA and certain glycoproteins, sugars or oligo-saccharides that inhibit cell-cell and cell-matrix interaction. In related embodiments, the non-desired preservative is EDTA. In other related embodiments; the non-desired preservatives are certain glycoproteins, sugars or oligo-saccharides that inhibit cell-cell and cell-matrix interaction.

In another aspect, the media composition of the invention contains one or more preservatives that do not affect viability or function of cell, sperm, oocyte, embryo or tissues. Examples of preservatives that do not affect viability or function of cell, sperm, oocyte, embryo or tissues include, without limit, boric acid, ascorbic acid, sodium borate, methyl paraben or a combination thereof. In certain related embodiments, the concentration of desirable preservative is between 0.00001% and 10%, preferably between 0.0001% and 5%, and most preferably between 0.001% and 1%.

In other embodiments, the media composition of the invention contains one or more other pharmaceutically useful agent including, without limit, anti-itch agents, anesthetic agents, estrogenic agents, antibiotic agents, steroids, therapeutic drugs, drug delivery vehicles and others, and including combinations thereof. Penicillin, streptomycin, gentamycin, or mixtures thereof are preferred antibiotics.

In certain embodiments, the media compositions of the invention may contain other implantation potential enhancing agents including, without limit, hyaluronan.

In certain other aspects, the media composition of the invention is in the form of a solution, powder, gel, foam, cream, jelly, or the like. Additionally, the compositions may also be packaged in sterile pre-filled bottles, applicators, tubes and other containers. In certain related embodiments, the composition is provided at a higher concentration such that dilution with one or more diluents, such as water, is performed prior to its use in an application.

In other aspects, the composition may be packaged in a kit. In a related embodiment, the kit may contain multiple such tubes. In another aspect, the kit may contain other items, such as, without limit, instruction sheets and the like.

In certain preferred aspects, the media compositions provided are non-spermicidal, sperm-friendly, oocyte-friendly and embryo-friendly. In a related embodiment, the media compositions provided increase fertilization-potential of sperm and of oocyte. In a related embodiment, the media compositions provided increase implantation potential of fertilized embryo.

In other embodiments, the media compositions of the invention are used as media for various steps and methods during artificial insemination or assisted reproduction techniques (ART) or to otherwise treat fertility issues in humans and other animals in vitro. Examples of such procedures include, without limit, sperm collection, sperm washing, sperm extension, oocyte collection, oocyte washing, in vitro fertilization and the like. For example, the compositions of the invention are used as medium for washing sperm, oocyte, embryo, cells or tissues. In another such embodiment, the compositions are used during the isolation of motile sperm from a sample. In another embodiment, the provided methods and compositions are useful in improving sperm-egg interactions, thereby increasing the chance of fertilization, in vivo and in vitro. In another such embodiment, methods and compositions are provided for improving the process of in vitro fertilization, artificial insemination or other fertility related treatments.

In certain embodiments, the media compositions of the invention are used as wash media in various steps of artificial insemination or assisted reproduction techniques (ART) or to otherwise treat fertility issues in humans and other animals in vitro. Examples of such procedures include, without limit, sperm collection, sperm washing, sperm extension, oocyte collection, oocyte washing, in vitro fertilization and the like. For example, the compositions of the invention can be used as medium for washing sperm, oocyte, embryo, cells or tissues. In certain other embodiments, the compositions are used for or during the isolation of motile sperm from a sample. In other embodiments, the provided methods and compositions are useful in improving sperm-egg interactions, thereby increasing the chance of fertilization, in vivo and in vitro. In another embodiment, methods and compositions are provided for improving the process of in vitro fertilization, artificial insemination or other fertility related treatments.

In other embodiments, the media compositions of the invention are used in vitro as culturing medium or extending medium for development of specialized cells, such as embryos and stem cells, using improvements in cell-cell binding, such as sperm-egg binding.

In another embodiment, methods and media compositions are provided for development of specialized cells, such as embryos and stem cells, using improvements in cell-cell binding, such as sperm-egg binding.

In other embodiments, methods for increasing survival of sperm, oocyte, embryo, cells, tissue are provided that include contacting them with a media composition of the invention. In certain other embodiments, methods for preserving the function of sperm, eggs and cells, for reducing the loss of sperm-function (and egg, oocyte and cell-function) and sperm, egg, oocyte & cell damage are provided. In other embodiments, methods for improving the function of sperm and cells are provided. In another embodiment, medium for storing sperm, oocyte, embryo or cells is provided.

In certain embodiments, the media compositions of the invention are used for collection of various biological samples, such as, without limit, cerebrospinal fluid, biopsy cells, biopsy tissue, cysts, tumors, saliva, stool, buccal swab, tissue, cells, fluid, blood, or a mixture thereof. In certain such embodiments, the compositions of the invention preserve viability of the collected biological material. In other embodiments, the compositions of the invention are useful as culturing or extending media immediately after collection of biological samples. In other such embodiments, the compositions of the invention are useful as culturing or extending media immediately for biological samples following their storage in a controlled temperature, heated, refrigerated, frozen or vitrified state.

In certain embodiments, the media compositions of the invention are used for collection of various microbiological flora (such as, without limit, bacteria, fungi, virus etc) from biological samples, such as, without limit, cerebrospinal fluid, biopsy cells, biopsy tissue, cysts, tumors, saliva, stool, buccal swab, cells, tissue, fluid, blood or a mixture thereof. In certain such embodiments, the compositions of the invention preserve viability of the collected micro flora. In other embodiments, the compositions of the invention are useful as culturing or extending media of the microflora immediately after collection of biological samples. In certain such embodiments, the compositions of the invention are useful as culturing or extending media of the microflora immediately for biological samples following their storage in a controlled temperature, heated, refrigerated, frozen or vitrified state. In other embodiments, the microflora collected using the media compositions of the invention are used in, without limit, development of assays, small molecules, therapeutics, high-throughput screens etc.

In certain aspects, the compositions and articles of the invention are prepared and/or produced by any method, including combining the active ingredients in the appropriate amounts and concentrations in a container and mixing. If need be, the mixtures are heated or cooled to aid in solvation of the ingredients. In certain embodiments, the compositions are sterile and a preferred method of sterilization is passing through a 0.2 micron filter. In certain embodiments, the compositions of the invention also have a high degree of clarity, preferably a turbidity of less than about 2, as measured with standard turbidimetric procedures known in the art.

In certain embodiments, the compositions of the invention can be useful for collection of various biological organs, such as, without limit: vagina, penis, kidney, lung, heart, liver, bone, skin and the like or a mixture thereof. In certain such embodiments, the compositions of the invention preserve viability of the collected organs. In other related embodiments, the compositions of the invention preserve physiological function of the collected organs. For example, the compositions of the invention may enhance the function of an organ post-transplantation, by reducing the rejection rate. In other embodiments, the compositions of the invention are useful as culturing or extending media immediately after collection of organs. In certain such embodiments, the compositions of the invention are useful as culturing or extending media immediately for organs following their storage in a controlled temperature, room temperature, heated, body temperature, refrigerated, frozen or vitrified state. In other related embodiments, the compositions of the invention are useful as transportation solutions for organs. In certain embodiments, the compositions of the invention improve the viability and/or physiological function of organs during culture, during storage, transportation and during in vivo and in vitro use. In certain embodiments, the sample is obtained from an animal, such as human, bovine, canine, equine, porcine, ovine, avian, rodent, or rare or exotic species, or is artificially generated.

In other embodiments, the compositions of the invention are used for medical treatments such as, without limit: wounds, rashes, burns, bruises, transplants and the like.

### III. Storage Media Compositions and Applications

In certain embodiments, a composition of the invention is used as storage media for preserving sperm, oocyte, embryo, cells or tissues during their storage in a controlled temperature, heated, refrigerated, frozen or vitrified state, comprising one or more viability-maintaining agents in a storage solution base. The composition of the invention preferably has a pH, osmolality and viscosity in a suitable range for maintaining or enhancing cell function. In certain aspects, the composition of the invention comprises agents that enhance physiological function. In certain such embodiments, the agents that enhance physiological function comprise sperm activation agents, energy-boosting agents, scavengers, fertilization facilitators or a mixture thereof. In certain embodiments, compositions of the invention are free or substantially free certain preservatives and buffers that affect viability or function of cell, sperm, oocyte, embryo or tissues. Examples of such preservatives include, without limit, EDTA, cyclic-RGD peptide and certain glycoproteins, sugars or oligo-saccharides that inhibit cell-cell and cell-matrix interaction.

In certain other aspects, the composition of the invention can be used as storage media for preserving sperm, oocyte, embryo, cells or tissues during their storage in a controlled temperature, heated, refrigerated, frozen or vitrified state, comprising one or more that enhance physiological function in a storage solution base. The compositions of the invention preferably have a pH, osmolality and viscosity in a suitable range for preserving cells or tissues. In other embodiments, the agents that enhance physiological function comprise sperm activation agents, energy-boosting agents, scavengers, fertilization facilitators, or a mixture thereof. In another embodiment, the compositions of the invention further comprise viability-maintaining agents. In other embodiments, compositions provided are free or substantially free of certain preservatives and buffers that affect viability or function of cell, sperm, oocyte, embryo or tissues. Examples of such preservatives include, without limit, EDTA, cyclic-ROD peptide and certain glycoproteins, sugars or oligo-saccharides that inhibit cell-cell and cell-matrix interaction.

In certain embodiments, storage solution base comprises a balanced salt solution that includes, but is not limited to, IMDM, DMEM, RPMI media, Tyrode's buffered salts, TALP, HTF, CZB, T6, Ham's F12, Earle's buffered salts, BWW, Earle's MTF, KSOM, SOF, and the like. Composition of each of these balanced salt solutions is well known in the art and many are commercially available (for example, from ATCC, Fisher Scientific, Invitrogen, VitroLife etc.). In another embodiment, buffering agents, such as TRIS, PIPES, HEPES are further added to these solutions. Additionally, certain other agents, such as Trehalose, DMSO, ethanol, other alcohols, glycerol, ethylene glycol, propylene glycol, hydroxypropyl methylcellulose, serum, albumin, gelatin, vitamins, minerals, amino acids, nucleotides, sucrose, other sugars and the like, may also be added to the balanced salt solutions. The composition comprises between 50% and 99.999% balanced salt solution, preferably between 75% and 99.99% balanced salt solution, and most preferably between 80% and 93% balanced salt solution. In certain embodiments, the preferred pH of the composition is in the range of 5.0 and 9.0, preferably between 7.0 and 8.5 preferably between 7.8 and 8.2 and preferably about 7.35; preferred osmolality is between 200 and 700 mOsm/kg, preferably between 250 and 500 mOsm/kg, preferably between 300 and 350 mOsm/kg and preferably about 320 mOsm/kg; and preferred viscosity, expressed as ratio with the viscosity of a balanced salt solution such as phosphate buffered saline (PBS), is between 1.0 and 5.0, preferably between 1.0 and 4.0, and most preferably between 1.0 and 3.0. In certain related embodiments, pH buffering agents comprise phosphate salts, borate salts, citrate salts, ascorbate salts, carbonate salts, bicarbonate salts, or a mixture thereof.

Sodium hydroxide is preferably added to adjust the pH. In certain related embodiments, osmolytes comprise sodium ions, potassium ions, inositol, betaine, sorbitol, peptides or glutamine. In certain such embodiments, the concentration of the osmolyte potassium ions is low, between 0.001 micromolar (μM) and 12.5 millimolar (mM), preferably between 0.1 μM and 10 mM, and most preferably between 10 μM and 5 mM.

In certain embodiments, the preferred viability-maintaining agent is ionic, and the preferable ions are calcium or magnesium. In certain embodiments, the composition comprises one or more viability-maintaining agents. In certain such embodiments, the provided concentration of viability-maintaining agent is between 0.001 micromolar (μM) and 1 molar (M), preferably between 0.01 millimolar (mM) and 10 mM, and most preferably between 0.1 mM and 5 mM. In certain other embodiments, the preferred viability-maintaining agent is selected from carbon monoxide, carbon dioxide, nitric oxide or a mixture thereof.

In certain embodiments, the preferred sperm activation agent is ionic, and the preferable ions are calcium, magnesium, manganese, or bicarbonate. In certain such embodiments, the composition comprises one or more sperm activation agents. In certain such embodiments, the provided concentration of sperm activation agent is between 0.001 micromolar (μM) and 1 molar (M), preferably between 0.01 μM and 50 mM, and most preferably between 1 μM and 30 mM. In certain embodiments, the preferred sperm activation agent is ionic calcium and the provided concentration of calcium is preferably between 10 μM and 10 mM, and preferably between 500 μM and 5 mM. In other such embodiments, the preferred sperm activation agent is ionic magnesium and the provided concentration of calcium is preferably between 10 μM and 10 mM, and preferably between 500 μM and 5 mM. In yet other such embodiments, the preferred sperm activation agent is bicarbonate ion and the provided concentration of bicarbonate ion is preferably between 100 μM and 50 mM, and most preferably between 10 mM and 30 mM. In other embodiments, the preferred sperm activation agent is cyclic AMP, caffeine, aspirin, carbon monoxide or a mixture thereof. In related embodiments, the preferred sperm activation agent is caffeine. In other embodiments, the preferred sperm activation agent is selected from hyaluronidase (such as PH-20), albumin, high-density lipoprotein, progesterone or a mixture thereof.

In certain embodiments, the preferred energy-boosting agent is ATP, fructose, glucose, pyruvate, lactose, lactate or a mixture thereof. In certain related embodiments, the concentration of energy-boosting agent is between 0.0001 μM and 1 M, preferably between 0.01 μM and 100 mM, and most preferably between 1 μM and 25 mM.

In certain embodiments, the preferred scavenger is vitamin E, Vitamin C, niacin, riboflavin, niacinamide or a mixture thereof. In related embodiments, the concentration of scavenger is between 0.0001 μM and 100 mM, preferably between 0.01 μM and 10 mM, and most preferably between 1 μM and 1 mM.

In certain embodiments, the preferred fertilization facilitator is magnesium ions, manganese ions, bicarbonate ions, hyaluronidase, progesterone, panthenol, caffeine, L-carnitine, cyclic-AMP or a mixture thereof. In related embodiments, the concentration of fertilization facilitator is between 0.0001 μM and 100 mM, preferably between 0.01 μM and 10 mM, and most preferably between 1 μM and 2 mM.

In another aspect, the compositions of the invention are free or substantially free of preservatives that affect viability or function of cell, sperm, oocyte, embryo or tissues. Examples of such non-desired preservatives include, without limit, EDTA and certain glycoproteins, sugars or oligo- and poly-saccharides that inhibit cell-cell and cell-matrix interaction. In certain related embodiments, the detrimental preservative is EDTA. In certain other related embodiments, the detrimental preservatives are certain glycoproteins, sugars or oligo- and poly-saccharides that inhibit cell-cell and cell-matrix interaction.

In yet another aspect, the compositions of the invention may contain preservatives that do not affect viability or function of cell, sperm, oocyte, embryo or tissues. Examples of the preservatives that do not affect viability or function of cell, sperm, oocyte, embryo or tissues include, without limit, boric acid, ascorbic acid, sodium borate, methyl paraben or a combination thereof. In related embodiments, the concentration of desirable preservative is between 0.00001% and 10%, preferably between 0.0001% and 5%, and most preferably between 0.001% and 1%.

In another aspect, the compositions of the invention may contain “protective agents” that reduce the loss of viable cells, tissues or organs during their storage in a controlled temperature, heated, refrigerated, frozen or vitrified state. Examples of the preferred protective agents include, without limit, hydroxypropyl methylcellulose, DMSO, albumin, serum, glycerol, trehalose, PCAGH, poly-saccharides, carbon monoxide, carbon dioxide, glycoproteins or a combination thereof. In certain such embodiments, the concentration of desirable preservative is between 0.01% and 50%, preferably between 0.1% and 25%, and most preferably between 1% and 10%.

In certain embodiments, the compositions of the invention reduce the loss of viable cells, tissues or organs during storage at low temperatures. In certain such embodiments, the protective agents are cryoprotectants that preserve the integrity of cellular structures and cellular function during low temperature storage. In Certain such embodiments, the cryoprotectancts preserve the integrity of cellular structures and cellular function during the cooling or warming of cells, tissues or organs. In certain embodiments the cryoprotectants comprise any of DMSO, glycerol, trehalose, albumin, serum, hypromellose, carbon monoxide, carbon dioxide, PCAGH, polysaccharides, glycoproteins, propylene glycol, ethylene glycol, formamide, N,N-dimethylformamide, glucose, sucrose, lactose, dextrose, raffinose, hydroxyethyl starch, gluconate, lactobionate, chondroitin sulfate, anti-freeze proteins, polyglycerol, polyvinyl alcohol, polyvinylalcohol oligomers, polyglycerol, polyvinylpyrrolidone or any combination thereof.

In other embodiments, the compositions of the invention may contain other pharmaceutically useful substances including, without limit, anti-itch agents, anesthetic agents, estrogenic agents, antibiotic agents, steroids, therapeutic drugs, drug delivery vehicles and others, and including combinations thereof. Penicillin, streptomycin, gentamycin, or mixtures thereof are preferred antibiotics.

In certain embodiments, the compositions of the invention contain other implantation potential enhancing agents including, without limit, hyaluronan.

In certain other aspects, the composition of the invention may be in the form of a solution, gel, foam, cream, jelly, or the like. Additionally, the compositions can be packaged in sterile pre-filled, applicators, tubes and other containers. In certain embodiments, the composition is provided at a higher concentration such that dilution with one or more diluents, such as water, is performed prior to its use. In certain aspects, the composition may be packaged in a kit. In certain related embodiments, the kit may contain multiple such tubes. In related embodiments, the kit may contain other items, such as, without limit, instruction sheets and the like.

In another aspect, the media compositions provided are non-spermicidal, sperm-friendly and increase fertilization-potential. In certain other embodiments, the media compositions provided increase the implantation potential of the embryo.

In other embodiments, the compositions of the invention are used as storage media during artificial insemination or assisted reproduction techniques (ART) or to otherwise treat fertility issues in humans and other animals. Examples include, without limit, sperm storage, oocyte storage, cell storage, embryo storage, tissue storage and the like.

In certain embodiments, the compositions and methods of the invention are useful in reducing the loss of viable and/or functional sperm, oocyte, embryo, cells or tissues during their storage in a controlled temperature, heated, refrigerated, frozen or vitrified state.

In certain embodiments, the compositions and methods of the invention are used for increasing the viability of sperm, oocyte, embryo, cells or tissues during their storage in a controlled temperature, heated, refrigerated, frozen or vitrified state.

In other embodiments, the compositions and methods of the invention are used for increasing the number of functional sperm, oocyte, embryo, cells or tissues during their storage in a controlled temperature, heated, refrigerated, frozen or vitrified state.

In other embodiments, methods for increasing survival of sperm, oocyte, embryo, cells, tissues are provided that include contacting them with a composition of the invention. In related embodiments, methods for preserving the function of sperm, eggs and cells, for reducing the loss of sperm-function (and egg, oocyte and cell-function) and sperm, egg, oocyte & cell damage are provided. In other embodiments, methods for improving the function of sperm and cells are provided. In certain embodiments, medium for storing sperm, oocyte, embryo or cells is provided. In certain related embodiments, medium and methods for sperm-banking, oocyte-banking, embryo-banking or cell-banking are provided. In other embodiments, the composition of the invention may also be used to coat tissues, surfaces and synthetic polymers, such as penile sheaths. In related aspects, lubricated sheath designs are also provided.

In certain embodiments, the storage media compositions of the invention are used as compositions for collection of various biological samples, such as, without limit, cerebrospinal fluid, biopsy cells, biopsy tissue, cysts, tumors, saliva, stool, buccal swab, tissue, cells, fluid, blood, or a mixture thereof. In certain such embodiments, the compositions of the invention preserve viability of the collected biological material. In other embodiments, the compositions of the invention are useful as culturing or extending media immediately after collection of biological samples. In other related embodiments, the compositions of the invention are useful as culturing or extending media immediately for biological samples following their storage in a refrigerated, frozen or vitrified state. In other embodiments, the compositions of the invention are useful as storage media for storage of biological samples in a controlled temperature, heated, refrigerated, frozen or vitrified state.

In certain embodiments, the storage media compositions of the invention are used for collection of various microbiological flora (such as, without limit, bacteria, fungi, virus etc) from biological samples, such as, without limit, cerebrospinal fluid, biopsy cells, biopsy tissue, cysts, tumors, saliva, stool, buccal swab, cells, tissue, fluid, blood or a mixture thereof. In certain such embodiments, the compositions of the invention preserve viability of the collected micro flora. In other related embodiments, the compositions of the invention are useful as culturing or extending media of the micro flora immediately after collection of biological samples. In related embodiments, the compositions of the invention are useful as culturing or extending media of the micro flora immediately for biological samples following their storage in a refrigerated, frozen or vitrified state. In other embodiments, the compositions of the invention are useful as storage media for storage of micro flora in a controlled temperature, heated, refrigerated, frozen or vitrified state. In another embodiment, the micro flora collected using the compositions are used in, without limit, development of assays, small molecules, therapeutics, high-throughput screenings etc.

In another embodiment, the compositions and articles of the invention are generally prepared and/or produced by any method, including combining the active ingredients in the appropriate amounts and concentrations in a container and mixing. If need be, the mixtures are heated or cooled to aid in solvation of the ingredients. The compositions are preferably sterile and the most preferred method of sterilization is passing through a 0.2 micron filter. In certain embodiments, the compositions of the invention also have a high degree of clarity, preferably a turbidity of less than about 2, as measured with standard turbidimetric procedures known in the art.

In certain embodiments, the compositions of the invention are used for collection of various biological organs, such as, without limit, vagina, penis, kidney, lung, heart, liver, bone, skin and the like or a mixture thereof. In certain such embodiments, the compositions of the invention preserve viability of the collected organs. In certain related embodiments, the compositions of the invention preserve physiological function of the collected organs. For example, the compositions of the invention may enhance the function of an organ post-transplantation, by reducing the rejection rate. In certain embodiments, the compositions of the invention are useful as culturing or extending media immediately after collection of organs. In certain related embodiments, the compositions of the invention are useful as culturing or extending media immediately for organs following their storage in a room temperature, heated, body temperature, refrigerated, frozen or vitrified state. In certain embodiments, the compositions of the invention are useful as transportation solutions for organs. In certain such embodiments, the compositions of the invention improve the viability and/or physiological function of organs during culture, during storage, transportation and during in vivo and in vitro use. In certain aspects, the sample is obtained from animals, including human, bovine, canine, equine, porcine, ovine, avian, rodent or rare and exotic species or is artificially generated.

In certain embodiments, the compositions of the invention are used during medical treatments for, without limit, wounds, rashes, burns, bruises, transplants and the like.

In certain embodiments, the compositions of the invention may include any or more of the components listed in Table 2. In certain embodiments, the percentage of any such component in the composition falls within the range of values indicated in Table 2. In certain such embodiments, any component listed in the table may be substituted by a (different) salt thereof and/or any suitable solvate and/or hydrate of that component or salt thereof. For example, KCI, as a source of potassium ions as described above, may be replaced by another suitable potassium salt, such as KOH, KHCO₃, KBr, etc., or by a hydrate of such a salt (e.g., KHCO₃ sesquihydrate) in an amount that provides an equivalent concentration of potassium ions in the composition. Similarly, an antioxidant may be replaced by a functionally equivalent amount of any other antioxidant discussed above, etc.

TABLE 2

| Component | High | Low |
|---|---|---|
| Hypromellose (lubricant, moisturizer) | 0.750% | 2.000% |
| NaCl (for maintaining osmolality, Na⁺ source) | 0.700% | 1.000% |
| glycerol (lubricant, moisturizer, for maintaining osmolality) | 0.125% | 2.000% |
| Na₂HPO₄ (anhydrous) (buffer, Na⁺ source) | 0.100% | 0.500% |
| NaH₂PO₄ (anhydrous) (buffer, Na⁺ source) | 0.010% | 0.100% |
| KCl (for maintaining osmolality, K⁺ source) | 0.001% | 0.050% |
| DL-α-tocopherol acetate (antioxidant) | 0.001% | 0.100% |
| MgCl₂•6H₂O (Mg⁺⁺ source) | 0.002% | 0.050% |
| CaCl₂•2H₂O (Ca⁺⁺ source) | 0.001% | 0.050% |
| methyl-4-hydroxybenzoate (methyl paraben) (lubricant, anti-bacterial, preservative) | 0.001% | 0.050% |
| Propyl paraben (lubricant, anti-bacterial, preservative) | 0.001% | 0050% |
| PLURONIC-127 (lubricant) | 0.010% | 1.000% |
| cyclic AMP (sperm activation agent, fertilization facilitator) | 0.005% | 0.050% |
| HDL (sperm activation agent, fertilization facilitator) | 0.010% | 0.100% |
| Panthenol (sperm activation agent, fertilization facilitator) | 0.100% | 2.000% |
| caffeine (sperm activation agent, fertilization facilitator) | 0.005% | 0.020% |
| Hyaluronidase (sperm activation agent, fertilization facilitator) | 0.001% | 0.050% |
| tocopherol succinate (antioxidant) | 0.001% | 0.100% |
| tocopherol nicotinate (antioxidant) | 0.001% | 0.100% |
| ascorbic acid (vitamin c) (antioxidant, preservative) | 0.001% | 0.100% |
| sodium bicarbonate (buffer, sperm activation agent, | 0.020% | 0.500% |
| fertilization facilitator, Na⁺ source) | | |
| propylene glycol (moisturizer, lubricant, for maintaining | 0.125% | 2.000% |
| osmolality) | | |
| citric acid (buffer, antioxidant, preservative) | 0.001% | 0.050% |
| lactose (energy-boosting agent, sperm activation agent, | 0.010% | 0.100% |
| fertilization facilitator) | | |
| sodium lactate (energy-boosting agent, sperm activation | 0.010% | 0.100% |
| agent, fertilization facilitator, Na⁺ source) | | |
| sodium pyruvate (energy-boosting agent, sperm activation | 0.005% | 0.020% |
| agent, fertilization facilitator, Na⁺ source) | | |
| glucose (energy-boosting agent, sperm activation agent, | 0.250% | 2.000% |
| fertilization facilitator) | | |
| sodium borate (buffer, preservative, Na⁺ source) | 0.010% | 0.100% |
| boric acid (buffer, preservative) | 0.010% | 0.050% |
| sodium citrate (buffer, antioxidant, preservative, Na⁺ | 0.100% | 0.400% |
| source) | | |
| sorbic acid (buffer, preservative) | 0.010% | 0.100% |
| potassium sorbate (buffer, preservative, K⁺ source) | 0.010% | 0.100% |
| calcium sorbate (buffer, preservative, Ca⁺⁺ source) | 0.010% | 0.100% |
| sodium sorbate (buffer, preservative, Na⁺ source) | 0.010% | 0.100% |
| manganese chloride (Mn⁺⁺ source) | 0.002% | 0.050% |

### IV. Sheaths

A condom generally refers to a receptacle structured for collecting semen from a penis, as described in the U.S. Manual of Patent Classification 604/349. A condom can be placed over the penis or an alternate design can be inserted within the vagina. A condom is usually flexible and is shaped and designed so as to fit around the penis to receive emitted semen. It is generally shaped as a tube-like structure extending from an open end to a closed end, with an elongated portion in the middle. The condom has an inner and an outer surface.

In certain embodiments, the present invention is directed towards articles similar in composition and structure to a condom, but that do not capture all or most of sperm or semen emitted during or after coitus. In a preferred aspect, the device, herein termed a sheath, does not prevent pregnancy. In a further embodiment, the sheath is coated with a lubricant that aids in the process of fertilization.

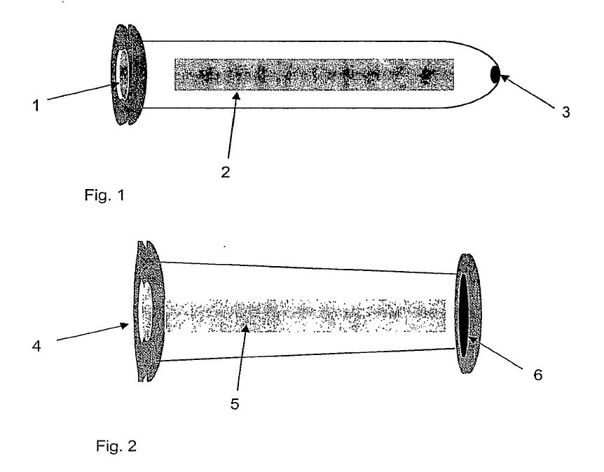

In certain aspects of the invention, the sheath is designed for placement over the penis (FIG. 1) or placement within the vagina (FIG. 2). In certain embodiments, the sheath of the invention has one or more holes present in the closed end that allow semen or sperm to escape during or after coitus (FIG. 1-3 & FIG. 2-6). In certain other embodiments, the lubricated sheath has one or more holes in the elongated portion of the tube-like structure that allow semen or sperm to escape during or after coitus. In certain other aspects, the sheath of the invention may have holes present in both the closed end and in the elongated portion. In certain aspects of the invention, the sheath is adapted for delivery of sperm directly, to the cervical opening, e.g., for directing sperm towards the opening and/or facilitating fertilization (reference: U.S. Pat. No. 5,857,959).

In certain embodiments, the external surface of the elongated portion of the tube-like portion of the sheath of the invention is coated with a lubricating composition (FIG. 1-2 & FIG. 2-5). In certain embodiments the internal surface of the tube-like portion of the sheath of the invention is coated with a lubricating composition (FIG. 1-1 & FIG. 2-4). In certain such embodiments, the lubricant for the sheath is non-spermicidal, and may be any composition described above in the invention.

In certain embodiments, the lubricated sheath of the invention is prepared and/or produced in analogy with methods known in the art for manufacturing condoms, including lubricated condoms. Various methods of manufacturing condoms are well known in the art, such as, without limit, dip-casting. In certain embodiments, the sheath is manufactured and sold in a rolled configuration. In certain embodiments, a sheath is made of thin, flexible, natural or synthetic elastic material, such as, without limit, latex, polyurethane, rubber, or rubber-like material. In certain embodiments, the sheath is packaged individually, with or without a lubricant, in a sealed pouch. In certain embodiments, the holes in the sheath are formed during the formation process. Examples of various manufacturing process steps, without limit, are dusting of condoms, rolling around thickened ring at the open end of the condom that leaves the generally closed end forming a cup within the circumference of the ring. In certain embodiments, the holes are created in the sheaths after the formation of the sheath is complete. In certain embodiments, the holes are created in the sheaths prior to their use.

The invention now being generally described, it will be more readily understood by reference to the following examples which are included merely for purposes of illustration of certain aspects and embodiments of the present invention, and are not intended to limit the invention.

### EXAMPLES

### Example 1

An exemplary composition related to the invention was prepared by the following procedure. First, a balanced salt solution comprising phosphate buffered saline (PBS) was prepared using the following protocol and ingredients:

| | |
|---|---|
| Na₂HPO₄ (anhydrous) | approx. 1.09 g |
| NaH₂PO₄ (anhydrous) | approx. 0.32 g |
| NaCl | approx. 9 g |
| Mix in distilled water to dissolve and adjust pH to 8.0. | |
| Add distilled water to bring the final volume to | approx. 1000 mL |

Viability-maintaining agents in the form of divalent calcium and magnesium ions were added to the PBS solution to achieve a final concentration of approx. 1 mM in each.

A base lubricant, hydroxypropyl methyl cellulose (hypromellose), was added in the following amounts and the resultant mixtures were gently mixed to obtain a solution for each composition:

Composition 1=0.5% (w/v) hypromellose
Composition 2=0.75% (w/v) hypromellose

### Example 2

An additional exemplary composition related to the invention was prepared by the following procedure. First, a base balanced salt solution comprising phosphate buffered saline (PBS++) was prepared using the following protocol and ingredients:

### Composition 3

### Solution A:

| | |
|---|---|
| Na₂HPO₄ (anhydrous) | approx. 0.234 g |
| NaH₂PO₄ (anhydrous) | approx. 0.048 g |
| Add distilled water to bring the final volume to | approx. 5.0 mL |

### Solution B:

| | |
|---|---|
| CaCl₂ 2 H₂O | approx. 0.013 g |
| KCl | approx. 0.026 g |
| MgCl₂ 6 H₂O | approx. 0.018 g |
| NaCl | approx. 0.80 g |
| Add distilled water to bring the final volume to | approx. 5.0 mL |

Solutions A and B were combined, the pH adjusted to 7.35, and the final volume brought up to 100 mL with distilled water. To this solution was added 1.5% (w/v) hypromellose, 0.01% (w/v) methylparaben, 0.02% (v/v) DL-a-tocopherol acetate, and 0.5% (v/v) glycerol and the resultant mixture was gently mixed to obtain a solution of composition 3.

### Example 3

Flow cytometric (FCM) analysis of sperm viability: The number of dead spermatozoa was measured according to a protocol in the published literature (24). Briefly, thawed spermatozoa were adjusted to a concentration of 1.0×10⁶ spermatozoa/mL in phosphate buffered saline (PBS) and washed by centrifugation. Washed spermatozoa were incubated with 3 mL of various lubricant compositions, including KY lubricant, Pre-Seed Lubricant (Bio-Origyn, LLC), Composition 1, Composition 2 and Composition 3 for 20 minutes. Afterwards, the spermatozoa were washed once, resuspended in PBS and incubated with annexinV-fluorescein isothiocyanate (FITC) solution (Pharmingen, San Diego, Calif.) and propidium iodide (PI) (Pharmingen, San Diego, Calif.) in the dark. The spermatozoa were washed once and the level of sperm viability was analyzed by four-color FCM analysis on a FACSort (Becton Dickinson, Mountain View, Calif.). The sperm population was gated using forward-angle light scatter to exclude debris and aggregates. A minimum of 10,000 individual spermatozoa were examined in each assay at a flow rate of <100 cells/s. The excitation wavelength was 488 nm supplied by an argon laser at 250 mW. Green (FITC-derived) fluorescence was measured using a 530 nm filter and the red fluorescence (PI) with a 610 nm filter. The percentage of P1 positive cells (dead spermatozoa) were calculated using FACSCaliber program (Becton Dickinson, Mountain View, Calif.) and the relative number of dead spermatozoa determined with each lubricant composition was calculated, with the percentage of dead spermatozoa determined using K-Y Jelly arbitrarily assigned a value of 100%. Results are presented in Table I.

Various patent and literature references are cited in this document. Each of these references is incorporated herein by reference in its entirety as if each were individually designated for incorporation.

Those skilled in the art will recognize, or be able to ascertain using no more than routine experimentation, many equivalents to the specific embodiments of the invention described herein. Such equivalents are intended to be encompassed by the following claims.

### REFERENCES

1. S. Greaves and C. Wang. A fertile field. Nat Cell Biol. 2002 October; 4 Suppl:S2
2. A. Glasier. Contraception—Past and future. Nat Cell Biol. 2002 October; 4 Suppl:S3-S6
3. P. M. Wassarman. Channels of communication in the ovary. Nat Cell Biol. 2002 October; 4 Suppl:S7-S9
4. L. R. Fraser A R T: Boon or bane? Nat Cell Biol. 2002 October; 4 Suppl:S10-S13
5. R. M. L. Winston and K. Hardy. Are we ignoring potential dangers of in vitro fertilization and related treatments? Nat Cell Biol. 2002 October; 4 Suppl:S14-S18
6. G. P. Schatten. Safeguarding ART. Nat Cell Biol. 2002 October; 4 Suppl:S19-S22
7. R. A. Charo, J. D. Children by choice: reproductive technologies and the boundaries of personal autonomy. Nat Cell Biol. 2002 October; 4 Suppl:S23-S28
8. P. Katz, R. Nachtigall and J. Showstack. The economic impact of the assisted reproductive technologies. Nat Cell Biol. 2002 October; 4 Suppl:S29-S32
9. R. M. Sharpe and S. Franks. Environment, lifestyle and infertility—an inter-generational issue. Nat Cell Biol. 2002 October; 4 Suppl:S33-S40
10. M. M. Matzuk and D. J. Lamb Genetic dissection of mammalian fertility pathways. Nat Cell Biol. 2002 October; 4 Suppl:S41-S49
11. M. D. Champion and R. S. Hawley. Playing for half the deck: the molecular biology of meiosis. Nat Cell Biol. 2002 October; 4 Suppl:S50-S56
12. J. P. Evans and H. M. Florman. The state of the union: the cell biology of fertilization. Nat Cell Biol. 2002 October; 4 Suppl:S57-S63
13. Butler D. The fertility riddle. Nature. 2004 Nov. 4; 432(7013):38-9.
14. Powell K. Skeptics demand duplication of controversial fertility claim. Nat Med. 2005 September; 11(9):911.
15. Kirichok Y, Navarro B, Clapham D E. Whole-cell patch-clamp measurements of spermatozoa reveal an alkaline-activated Ca2+ channel. Nature. 2006, 9; 439(7077):737.
16. Powell K. Fertility treatments: Seeds of doubt. Nature. 2003 Apr. 17; 422(6933):656-8.
17. Chen Y, Cann M J, Litvin T N, lourgenko V, Sinclair M L, Levin L R, Buck J. Soluble adenylyl cyclase as an evolutionarily conserved bicarbonate sensor. Science. 2000 Jul. 28; 289(5479):625-8.
18. Goldenberg R L, White R. The effect of vaginal lubricants on sperm motility in vitro. Fertil Steril. 1975 September; 26(9):872-3.
19. Kutteh W H, Chao C H, Ritter J O, Byrd W. Vaginal lubricants for the infertile couple: effect on sperm activity. Int J Fertil Menopausal Stud. 1996 July-August; 41(4):400-4.
20. Frishman G N, Luciano A A, Maier D B. Evaluation of Astroglide, a new vaginal lubricant: effects of length of exposure and concentration on sperm motility. Fertil Steril. 1992 September; 58(3):630-2.
21. Anderson L, Lewis S E, McClure N. The effects of coital lubricants on sperm motility in vitro. Hum Reprod. 1998 December; 13(12):3351-6.
22. Tagatz G E, Okagaki T, Sciarra J J. The effect of vaginal lubricants on sperm motility and viability in vitro. Am J Obstet Gynecol. 1972 May 1; 113 (1):88-90.
23. Visconti et al., Novel signaling pathways involved in sperm acquisition of fertilizing capacity, Journal of Reproductive Immunology, 2002, 53, 133-150.
24. Glander H J, Schaller J. Binding of annexin V to plasma membranes of human spermatozoa: a rapid assay for detection of membrane changes after cryostorage. Mol Hum Reprod. 1999 February; 5(2):109-15.

1. A topical lubricant composition, wherein the composition comprises:
(a) a non-spermicidal aqueous lubricant base for topically lubricating reproductive tissue, and
(b) a viability-maintaining agent for maintaining or increasing the viability of reproductive tissue, cells or both.

2. The topical lubricant composition of claim 1, further comprising:
(c) an agent that enhances physiological function for improving the potential of reproductive tissue, cells or both to perform their natural function.

3. A topical lubricant composition, wherein the composition comprises:
(a) a non-spermicidal aqueous lubricant base for topically lubricating reproductive tissue, and
(b) an agent that enhances physiological function for improving the potential of reproductive tissue, cells or both to perform their natural function.

4. The topical lubricant composition of claim 1, wherein the viability-maintaining agent maintains or increases the viability of sperm.

5. The topical lubricant composition of claim 1, wherein the viability-maintaining agent maintains or increases the viability of an egg.

6. The topical lubricant composition of claim 2, wherein the physiological function-enhancing agent improves the function of sperm.

7. The topical lubricant composition of claim 2, wherein the physiological function-enhancing agent improves the function of an egg.

8. The topical lubricant composition of claim 1, wherein the viability-maintaining agent is selected from calcium salts, sodium salts, potassium salts, magnesium salts, other ions, salts, lipids, small molecules, carbon monoxide, carbon dioxide, nitric oxide, nucleosides, nucleotides, sugars, peptides and proteins.

9. The topical lubricant of claim 8, wherein the viability-maintaining agent of the composition comprises a calcium or magnesium salt, or both.

10. The topical lubricant composition of claim 1, wherein the composition comprises an agent selected from a:
(a) sperm activation agent,
(b) energy-boosting agent,
(c) free-radical scavenger agent,
(d) fertilization facilitator agent, and
(e) embryo implantation facilitator agent.

11.-81. (canceled)
